(19) United States
(12) Patent Application Publication
(10) Pub. No.: US 2012/0149972 A1
(43) Pub. Date: Jun. 14, 2012

(54) Personalized, electronically-conveyed self-improvement messaging

(76) Inventor: Margaret Moore, Wellesley, MA (US)

(21) Appl. No.: 12/928,314

(22) Filed: Dec. 8, 2010

(51) Int. Cl. A61M 21/00

(52) U.S. Cl. 600/26

(57) ABSTRACT

The invention features a computer for fostering self-improvement in a person. The computer includes means for electronically conveying an audible message to the person. The message includes content that is generated by the person and that relates to the person's self-improvement, and includes phrases generated by the person. At least a portion of the message is an audible voice, the person's own voice or the voice of the person's coach family member, coach, or other trusted individual. Alternatively, the voice can be computer-generated. The computer can also include, or be operationally connected to, a monitor which displays messages to the person relating to the person's self-improvement.

### BACKGROUND OF THE INVENTION

Recently, researchers have used hand-held, mobile or desktop computers to “nudge” clients toward sustained engagement in behaviors which deliver self-improvement, most notably improved health and wellness. Gasser et al., in “Persuasiveness of a Mobile Lifestyle Coaching Application Using Social Facilitation” (2006) LNCS 3962 describe a Smartphone application that monitors the reporting by the client of intake of fruits and vegetables and physical activity, and automatically sends reminders in the absence of a report.

Timothy Bickmore of the Northeastern University College of Computer and Informational Science published an article, “What Would Jiminy Cricket Do? Lessons From the First Social Wearable” (2008), in which he describes a mobile Smartphone adapted to “detect when the user is at a point of decision making, or perhaps has just started to engage in an unhealthy behavior” (such as smoking, detected with a smoke detector) and in response, via a display screen, advises against carrying out the behavior. The initial studies concerned exercise promotion. The screen, as well as displaying text, included an animated face capable of expressing emotion (e.g., head nods).

The MIT Media Lab Personal Robots Group prepared a paper, “Robots at Home: Understanding Long-Term Human-Robot Interaction” (2009), Kidd and Breazeal, which describes robots that have once-or-twice daily interactions with overweight users cajoling them (preferably politely) to adopt behaviors that will aid in weight loss. The hardware and software are described in detail in Dr. Kidd's Ph.D. thesis, Kidd, “Designing for long-term human-robot interaction and application to weight loss,” Ph.D., Massachusetts Institute of Technology, February, 2008. The robots have heads with eyes, and also include, below the head, a touch screen for display and user input. Messages to the user can be in the form of the coach's voice, or text that appears on the screen. Messages can be scheduled for pre-arranged times, or can be transmitted in response to input from the user.

### SUMMARY OF THE INVENTION

The invention features a computer for fostering self-improvement in a person. The computer includes means for electronically conveying an audible message to the person. The message includes content that is generated by the person and that relates to the person's self-improvement, and includes phrases generated by the person. At least a portion of the message is an audible voice, the person's own voice or the voice of the person's coach family member, coach, or other trusted individual. Alternatively, the voice can be computer-generated. The computer can also include, or be operationally connected to, a monitor which displays messages to the person relating to the person's self-improvement.

By “computer” is meant any device that has electronic/digital computing capability and, preferably, digital word processing capability. Such devices include desktop computers, e.g., PCs and Macs, as well as mobile, hand-held devices such as Smartphones, Blackberrys®, iPhones®, and dedicated PDAs, which can take any form, including rectangular phone-shaped devices. In the prior art relating to message-conveying computers, such computers are sometimes referred to as “relational agents” or “RAs.” The more generic term “device” is used herein.

A central tenet underlying the invention is that, by employing content and phrases chosen by the user, along with the user's voice, the device taps into the user's desire to be his or her “best self,” awakens the user's parts of self which are future-oriented and are drowned out by the parts which want to do the expedient and more immediately appealing behavior.

The device also facilitates envisioning of the user's desires, hopes, dreams, and aspirations. The devices of the invention are intended to enable self-coaching and the building of autonomy, motivation, and confidence in the user.

Many people struggle with self-regulation of personal habits on a daily basis—making positive choices that best serve a person's future rather than choices that are expedient or provide pleasure in the moment and risk or damage the person's future. People make hundreds of choices every day in all areas of their lives and are often not mindful or purposeful in making the best choices—spending money, eating, drinking, exercising, listening, organizing, recharging, planning, connecting, savoring, appreciating, and on and on. People often start the day with great intentions toward making or sustaining positive changes in their lives. Then they become vulnerable to poor choices if they are mentally or physically tired, hungry, stressed, unsupported, worried, angry, sad, unsupported, or defiant in response to external pressure.

The self-improvement facilitated by the devices of the invention can be fitness/health-related, e.g., weight control, fitness, sleep issues, nutrition choices, and stress management. Other applicable areas of self-improvement include mental and emotional health (attention deficit disorder, obsessive-compulsive disorder, depression, anxiety, and the fostering of psychological resources including mindfulness, self-motivation, positive emotions, self-efficacy, optimism, resilience, and deployment of strengths and talents); management of medication or device-requiring medical conditions such as angina, diabetes, and hypertension; parenting (building autonomy and self-respect in family members); caregiving (building and maintaining a positive and resilient mindset in both caregiver and the person being cared for); managing finances; fostering spirituality; and nurturing relationships (e.g., fostering improved marriages, including anger management, conflict resolution).

Other applications involve self-regulation, self-development, self-help, and aiding performance in any of many areas: training for a race or developing new skills (playing chess, sailing, etc).

The self-generated message (often with the help of a coach) can relate to a life topic, a particular performance outcome, or behavior, skill, or psychological resource. The messages need not be in “command” form, but can be in the form of open-ended questions (e.g., “What do feel like doing now to get closer to your goal?”). The messages can also be closed: “Are you going to skip the elevator and take the stairs on your way home?” The timing of the messages is variable as well; some clients will feel pestered by too many message; others will welcome more. An end-of-day message that many clients are likely to respond wraps up the day: “What worked for you today? What did you learn? Name three things for which you feel grateful.”

Some of the messages can be set for delivery at fixed times during the day, while others can be triggered by the user, e.g., in a moment of weakness while reaching for a donut; the person touches an activating screen icon or computer button which delivers the message (e.g., “Do you want to pause for ten seconds before you pick that up?”).

The computer can also include means for storing the person's Personal Health Record, or means for accessing the person's Personal Health Record by synching with another computer in which the records are stored.

The computer can also include means for receiving oral or typed messages from the user to the computer for storage and later retrieval; the computer can also maintain a record of the messages conveyed by the computer to the user.

The computer can also include means enabling communication between the person and a health or wellness coach; this can be e-mail, Twitter, or voicemail; live (in person or by telephone or by phone capability included in the computer) coaching sessions can be incorporated into the self-improvement regimen of the computer user.

Potential markets are: chronic disease management, newly-diagnosed diseases with which the patient is unfamiliar, the workplace, the consumer (self-help), and the educational world lower schools and universities), and coaching (wellness, athletics, life, executive).

### EXAMPLES

### John

John is a client of a wellness coach, Coach Meg. John's doctor has told him that he has Metabolic Syndrome (large waist, high triglycerides, etc.) and that, if he wants to see his children graduate from college, he needs to lose sixty pounds and keep them off. Also, he needs to begin an exercise program immediately. The doctor put John in touch with Meg.

John and Meg agree that Meg will coach John in biweekly 45-minute wellness coaching sessions, using an Internet-based coaching system that Meg and her colleagues have developed and taught to other coaches under the banner of Meg's company, Wellcoaches Corporation; the system is described in part in Clark and Moore U.S. Pat. No. 7,376,700. In addition, to providing John with motivation and timely nudges to aid in reducing the intake of “bad” calories and increase physical activity, Meg and John together set up a mobile, handheld PDA (e.g., an iPhone) using known prior art software and hardware systems, e.g., those described in Kidd and Breazeal, supra, hereby incorporated by reference. John records his voice speaking messages of his choosing (sometimes with the aid of his coach); John has input into the content and phraseology of his own oral messages, which are stored in the device for later play-back.

John has the option of recording his own voice, or that of someone he chooses, such as his coach, a relative, friend, physician, or a teacher. He chooses to use his own audible, recorded voice. Meg helps him craft engaging phrases, keeping in mind the vision of his own “best self” urging him on, which he worked to define over the course of several coaching sessions. He has a good sense of humor so he chose phrases which are funny and not serious, and make him smile.

John settles on five phrases, each to be used in a different situation. Three are thrice-per-day scheduled voice messages (one to be played after dressing in the morning, a second before lunch, a third mid-afternoon). The other two are situational, and are activated by John pressing a prompt button on the keyboard or touch screen. Meg asks John if he ever gives himself pep talks, in his head or in front of the mirror, or out loud in the car. He says, yes, frequently; the recorded phrases derive from those self-pep talks. Even before trying the device, John understands that hearing the voice of his own best self will bring about self-motivation which, Meg says, is really the only kind that leads to changing for good.

The first message of the day, conveyed at 7 AM, says, in John's recorded voice, “Good morning. We're ready for a great breakfast of oatmeal, orange juice, and low-sodium smoked salmon, with side orders of no bacon, no Danishes, and no donuts.”

One of the situational messages, again in John's voice, is “Think about how good we're going to feel if you put down that candy bar and eat an apple instead.”

Within five minutes of hearing a message, John speaks into a microphone incorporated into the device, saying, “Got it.” (He has decided against the other response options Meg presented, including simply pressing a button labeled, “Got it.”)

At about eight PM every day, John uses the keyboard or touch screen of his device to record:
1. The date and time.
2. What food and drink he has consumed that day, and what he intends to consume between now and bedtime.
3. The amount and kind of exercise he's done that day.
4. His mood at mid-day (1-5).
5. His energy level mid-day (1-5).
6. His current mood (1-5).
7. His current energy level (1-5).
8. Changes he wishes to make to any of the recorded messages.

The device has been programmed to analyze John's usage patterns, just as his coach would track and provide encouraging feedback on recent history and progress. The device can send John messages every week in his own voice (pre-recorded to respond to success or setbacks) or the voice of his coach Meg based on such “feedback.” Thus, each week the device gives John a message on his screen (that is copied to Meg): You didn't respond to message seven “About time for your daily endoprphin-generating workout,” but you responded to almost all of the other messages throughout the day. John's voice says: “John—this is your higher self. Don't waste a moment beating yourself up. Just do it—something intense for ten minutes.”or: “Remember how great you feel after a mid-day workout. It leads to your best ideas and thinking.”

John has a 45-minute biweekly telephone coaching session with Meg, and during every fourth session they review the data stored in John's handheld device. They incorporate the data into the online goals/results page they maintain in the Wellcoaches web coaching platform, in which, among other things, John's long-term and short-term goals are stored and reviewed, and his progress, including weight loss, and peace of mind rating, are tracked. John also identifies the content for messages that he will record which celebrate his progress or cheer him on when a setback arises.

### Mary

Mary has Attention Deficit Disorder (ADD). Like many people with ADD, she is creative; lots of thing interest her, briefly. When she starts her work day, she generally has in mind several projects to work on, but as the day progresses she finds that she can't remember what they are, and doesn't have a clear vision for prioritizing the projects. Mary works in an open office, so she does not want auditory reminders; she opted for text messages, which she and Meg work on together, and in fact Meg signs some of them. Mary records the same information that John does, above; one might think that some of that recorded information is irrelevant to Mary, whose issue, ADD, is so different from John's, which involves, among other things, the management of his weight and exercise, but in fact much research demonstrates a connection between ADD symptoms and obesity. In fact, for every example, the same parameters are tracked, although there are additional ones for relationships and control of finances, for example.

In Mary's case, her self-messages are different from John's. The first of the day, after breakfast, is in her own “voice,” (it is a text message), that reads: “Mary, before you go to the office, look in the mirror and tell yourself that you look like you have your act together, then identify what 3-5 projects you are going to work on today, in order of priority. Promise yourself you'll follow through and remind yourself of your top three character strengths. After you've been in the office for ninety minutes, go for a walk and report, out loud to yourself, your progress.

Mary's second reminder arrives at 11:30, a text message from Meg: “Mary, have a great rest of the day. You have phone calls to return, some of which you wish you didn't have. Pick the one you most dread and return it first; then return the rest in decreasing order of difficulty. You'll have earned your lunch.

Mary's third automatic reminder is an after-work audio message from herself that says, “Keep it up. Relax tonight.”

There is one self-activated text message, usable at any time, a text message that Mary wrote that says, “TIME TO RETURN TO PLANET EARTH [symbol] !” in all capital letters. When she finds herself sitting at her disk daydreaming, she activates the message.

Mary's device contains not just the records of her days, but her personal health record, including the medications she is taking. She could have included medication reminders in the device, but she finds that doesn't need reminders for pills she takes during the day, a twice-per day stimulant and a once-daily hypertension pill.

Mary meets with Meg regularly to talk about progress—i.e., to have a coaching session, and tweak the messages to fit changed circumstances.

1. A computer for fostering self-improvement in a person, said computer including means for electronically conveying a message to said person, wherein said message:
a. contains content that is generated by the person and that pertains to the person's self-improvement,
b. includes phrases generated by the person, and
c. is at least in part conveyed in the audible voice of the person, another person, or a computer-generated voice

2. The computer of claim 1, wherein the computer includes or is operationally connected to a word-displaying monitor and a portion of said message-conveying means comprises readable phrases displayed on said monitor.

3. The computer of claim 1, wherein the self-improvement pertains to mental or physical wellness.

4. The computer of claim 3, wherein physical wellness pertains to weight-control.

5. The computer of claim 1, wherein the computer stores the person's Personal Health Record or includes means for accessing the person's Personal Health Record via synching with another computer.

6. The computer of claim 7, wherein the computer includes means for receiving messages from the person and means for storing both the person-generated messages and the messages conveyed to the person by the computer.

7. The computer of claim 1, wherein at least some of the computer-conveyed messages are automatically set to be conveyed at certain pre-determined times during the day.

8. The computer of claim 1, wherein the computer comprises means, e.g. e-mail, text Skype, Twitter, or voice, that enables the person to have a two-way interaction with a coach.
